(19) United States
(12) Patent Application Publication
(10) Pub. No.: US 2023/0000818 A1
(43) Pub. Date: Jan. 5, 2023

(54) CANNABICHROMENE AS A THERAPEUTIC MODALITY FOR COVID-19

(71) Applicant: AUGUSTA UNIVERSITY RESEARCH INSTITUTE, INC., Augusta, GA (US)

(72) Inventors: Babak BABAN, Augusta, GA (US); Krishnan DHANDAPANI, Evans, GA (US); Jack C. YU, Martinez, GA (US)

(73) Assignee: AUGUSTA UNIVERSITY RESEARCH INSTITUTE, INC., Augusta, GA (US)

(21) Appl. No.: 17/810,484

(22) Filed: Jul. 1, 2022

Related U.S. Application Data
(60) Provisional application No. 63/218,160, filed on Jul. 2, 2021.

(51) Int. Cl. A61K 31/352; A61K 9/00; A61P 31/14

(52) U.S. Cl. CPC A61K 31/352; A61K 9/0073; A61P 31/14

(57) ABSTRACT

Compositions and methods for treating or reducing symptoms of acute respiratory distress syndrome (ARDS) generally and COVID-19 infection in particular are provided herein. An exemplary method includes administering to the subject an effective amount of cannabichromene to reduce acute respiratory distress syndrome caused by ARDS generally and COVID-19 infection in particular.

### CROSS-REFERENCE TO RELATED APPLICATIONS

This application claims benefit of and priority to U.S. Provisional Application No. 63/218,160 filed on Jul. 2, 2021, and where permitted all of which are incorporated by reference in their entireties.

### STATEMENT REGARDING FEDERALLY SPONSORED RESEARCH

This invention was made with government support under NS110378 awarded by the National Institutes of Health. The government has certain rights in the invention.

### TECHNICAL FIELD OF THE INVENTION

This invention is generally related to compositions and methods of treating acute respiratory distress syndrome (ARDS).

### BACKGROUND OF THE INVENTION

COVID-19 pandemic has profoundly affected human life, inducing high patient morbidity and mortality while stressing health care systems worldwide. SARS-CoV-2, the highly infectious agent responsible for the COVID-19 pandemic, is a novel coronavirus that utilizes a glycosylated spike protein to enter human cells via the angiotensin-converting enzyme 2 (ACE2) receptor. The lung is a primary site of entry for SARS-CoV-2, as evidenced by massive pulmonary inflammation and development of acute respiratory distress syndrome (ARDS) (Saxena, S K., et al., Coronavirus Dis.; 2019:1 (2020)). ARDS is a serious inflammatory lung condition responsible for the highest rate of medical complications and mortality among critically ill patients (Gan, T., et al., Front Microbiol, 21; 9:3174 (2018)). In the case of viral respiratory infections, symptoms are usually mild, self-limiting, and confined to the upper airways. However, in more severe respiratory cases, as seen during the COVID-19 pandemic, the infection can affect the lower airways, causing ARDS, increased pulmonary vascular permeability, hypoxemia, increased coagulation, fibrinolysis factors, endothelial and epithelial damages (Ferguson, N D., et al., Intensive Care Med, 38(10):1573 (2019); Spadaro, S., et al., J Inflamm (London), 15; 16:1 (2019)). In patients with severe COVID-19, the transition to ARDS condition is mainly due to the occurrence of a cytokine storm and hyperinflammatory responses, including massive production of pro-inflammatory cytokines such as IL-6 and IL-1β, as well as infiltration of neutrophils and monocytes into the lung tissue (Spadaro, S., et al., J Inflamm (London), 15; 16:1 (2019); Ghadimi-Moghadam, A., et al., J Biomed Phys Eng, 10(2):241 (2020); Ye., Q., et al., J Infect, pii: S0163-4453(20)30165 (2020)). The cytokine storm contributes to diffuse alveolar damage, alveolar capillary leakage, severe hypoxaemia, intense pulmonary oedema and pulmonary fibrosis (Villar, J., et al., Chest, 155:587)2019). Currently, other than supportive measures there is no definitive cure for ARDS (Shen, C., et al., JAMA, March 27 (2020); Chaari, L., et al., EPMA J, 25:1-6 (2020)), illustrating the urgent need for creative and effective therapeutic modalities to treat this complex condition. Although the COVID-19 pandemic has deepened the understanding of ARDS pathophysiology, definitive therapy for ARDS continues to elude the medical and scientific community. If initiated early, combinatorial therapies such as corticosteroid and IL-6 inhibitor have shown beneficial effects in the management of ARDS (Harahwa, T A., et al., Acta Biomed., 91(4): e2020122,4 (2020); Hough, C L., et al., Clin Chest Med. 35(4):781-795 (2014)). However, there remains a need to develop alternative novel therapeutic targets for the prevention and treatment of ARDS persists.

Cannabinoids are naturally occurring compounds in Cannabis plants. Numerous studies suggest beneficial effects of cannabinoids in clinical settings. Of over 100 known cannabinoids, four, including tetrahydrocannabinol (Δ⁹-THC), cannabidiol (CBD), cannabinol (CBN), and cannabichromene (CBC), have attracted the most attention due to supporting evidence of their potential as therapeutic targets. Despite their structural differences, both CBD and CBC are non-psychoactive compounds with potential immunomodulatory effects and several other health benefits. More recently, in a set of preclinical studies we showed for the first time that CBD could ameliorate the symptoms of ARDS. Importantly, unlike THC and CBD, CBC is not categorized as a scheduled compound by U S Enforcement Agency (DEA), which may facilitate the use and assessment of CBC in clinical settings.

Numerous studies report that cannabinoids may function as immune modulators, limiting the adverse effects of inflammatory diseases (Pini, A., et al., Curr Drug Targets, 13(7):984 (2012)). Endocannabinoids are produced in the respiratory system and cannabinoids-induced bronchodialation suggest a significant therapeutic potential for cannabinoids in the treatment of respiratory diseases, including ARDS in case of patients with severe form of COVID-19 (Bozkurt, T E., Molecules, 24(24). pii: E4626 (2019)). Further, several reports suggest that CBC may act through the transient receptor potential cation channel subfamily A member 1 (TRPA1) as well as transient receptor potential cation channel subfamily V member 1 (TRPV1) with less affinity, likely affecting tissue homeostasis and immune balance as well as endocannabinoid cellular reuptake, and modulating the inflammatory responses. CBC has also been reported to be an agonist to the CB2 receptor, activation of which may further modulate inflammatory responses.

Apelin, an endogenous, multi-functional ligand for the G protein-coupled receptor, APJ, also serves as a second catalytic substrate for ACE2 (Chen L J, et al., Int J Hypertens. 2015: 5-10 (2015)). Apelin is generated from a 77-amino acid precursor and undergoes proteolytic cleavage to generate biological active fragments, including apelin-36, apelin-19 and apelin-13. An endogenous protective role was postulated for activation of the apelin/APJ axis (Apelinergic system) after lung injury, via proposed mechanisms including suppression of the immune activating transcription factor, NF-κB and inhibition of innate immune infiltration/activation via attenuated expression of CCL2, CCL3, CCL4, CCL7 and TNF-α (●Huang, S. et al., Clin Chim Acta. 456: 81-88 (2016)). Of interest, both apelin and APJ are widely expressed throughout the lung, heart, liver, gut, kidney and central nervous system (8Kawamata. Y. et al., Biochim Biophys Acta. 1538(2-3): 162-171 (2001)), spatially overlapping expression of the endocannabinoid system while interaction between the endocannabinoid system and apelin limits liver fibrosis (Melgar-Lesmes. P. et al., Cells. 8: 1311 (2019)).

Considering limited number of target-specific antiviral treatment and vaccination for COVID-19, it is absolutely exigent to have effective therapeutic modalities to reduce hospitalization and mortality rate as well as to improve COVID-19-infected patient outcomes. Specifically, it is of interest to find therapeutic modalities that are able to reverse the symptoms of ARDS towards a normal level.

Therefore, it is an object of the invention to provide compositions and methods of treating or reducing Acute respiratory distress syndrome and other inflammatory conditions associated with COVID-19.

### SUMMARY OF THE INVENTION

Disclosed herein are cannabinoid based compositions and methods of their use to treat or reduce symptoms associated with viral infections including but not limited to coronaviruses such as SARS-CoV-2 infection or COVID-19. Exemplary cannabinoid based compositions that can be used in the disclosed methods include, but are not limited to, tetrahydrocannabinols (THC), preferably delta-9-tetrahydrocannabinol and delta-8-tetrahydrocannabinol, cannabidiol (CBD), cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV) and cannabigerol monomethyl ether (CBGM), arachidonoylethanolamine (AEA), 2-arachidonoylglycerol (2-AG), 2-arachidonyl glyceryl ether (noladin ether), N-arachidonoyl dopamine (NADA), virodhamine (OAE) lysophosphatidylinositol (LPI), nabilone, rimonabant, JWH-073, CP-55940, dimethylheptylpyran, HU-210, HU-331, SR144528, WIN 55,212-2, JWH-133, levonantradol, and AM-2201 and combinations thereof. One embodiment provides administering an effective amount of a cannabichromene based composition, for example CBC, to a subject in need thereof to treat or reduce ARDS symptoms. In one embodiment, the disclosed cannabichromene compositions ameliorate the conditions associated with ARDS by reducing inflammation in the lung or airways, reducing inflammatory indices, limiting damage in the lung, and improving the functional capacity of airways.

Another embodiment provides a method of reducing inflammatory symptoms of COVID-19 by administering to a subject in need thereof an amount of cannabichromene effective to reduce inflammation in the subject including, but not limited to inflammation in the lung or airways.

Another embodiment provides a method of reducing ARDS in a subject in need thereof by administering to the subject an amount of cannabichromene effective to reduce inflammation in the subject. In some embodiments cannabichromene reduces the level of inflammatory cytokines including, but not limited to interleukin (IL)-2, IL-7, IL-6, IL-10, tumor necrosis factor (TNF), IFNγ, granulocyte colony-stimulating factor (G-CSF), monocyte chemoattractant protein-1 (MCP1; also known as CCL2), macrophage inflammatory protein 1 alpha (MIP1α; also known as CCL3), CXC-chemokine ligand 10 (CXCL10), C-reactive protein, ferritin, and D-dimers in blood upon SARS-CoV-2 infection. Of note, among the elevated inflammatory mediators, the blood IL-6 level is highly correlated with the disease mortality when COVID-19 survivors and non-survivors are compared, suggesting that fatal COVID-19 is characterized as a cytokine release syndrome (CRS) that is induced by a cytokine storm with high mortality (Shintaro Hojyo, et al., Inflamm Regen. 2020; 40: 37). The inflammatory cytokines can be circulating cytokines or pulmonary cytokines. In some embodiments cannabichromene treatment reduces inflammatory damage to the lungs and improves the functional capacity of the lungs. In one embodiment, the acute respiratory distress syndrome is caused by COVID-19.

Another embodiment provides a pharmaceutical composition containing an effective amount of a cannabinoid to reduce inflammation in a subject in need thereof. The pharmaceutical composition can be formulated for pulmonary administration, nasal administration, or aerosol administration. In one embodiment the cannabinoid is cannabichromene. In other embodiments, the pharmaceutical composition contains one or more cannabinoid selected from the group consisting of tetrahydrocannabinols (THC), preferably delta-9-tetrahydrocannabinol and delta-8-tetrahydrocannabinol, cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV), cannabigerol monomethyl ether (CBGM), arachidonoylethanolamine (AEA), 2-arachidonoylglycerol (2-AG), 2-arachidonyl glyceryl ether (noladin ether), N-arachidonoyl dopamine (NADA), virodhamine (OAE) lysophosphatidylinositol (LPI), nabilone, rimonabant, JWH-073, CP-55940, dimethylheptylpyran, HU-210, HU-331, SR144528, WIN 55,212-2, JWH-133, levonantradol, and AM-2201 and combinations thereof.

Still another embodiment provides a method of reducing pulmonary inflammation in a subject in need thereof by administering to the subject an effective amount of a cannabinoid to reduce the pulmonary inflammation. In one embodiment the cannabinoid is cannabichromene. In other embodiments, the cannabinoid is selected from the group consisting of tetrahydrocannabinols (THC), preferably delta-9-tetrahydrocannabinol and delta-8-tetrahydrocannabinol, cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV) and cannabigerol monomethyl ether (CBGM), arachidonoylethanolamine (AEA), 2-arachidonoylglycerol (2-AG), 2-arachidonyl glyceryl ether (noladin ether), N-arachidonoyl dopamine (NADA), virodhamine (OAE) lysophosphatidylinositol (LPI), nabilone, rimonabant, JWH-073, CP-55940, dimethylheptylpyran, HU-210, HU-331, SR144528, WIN 55,212-2, JWH-133, levonantradol, and AM-2201 and combinations thereof. In some embodiments, the subject is infected with a coronavirus including but not limited to SARS-CoV-2. In some embodiments, the subject has ARDS.

Yet another embodiment provides a method for treating or reducing a cytokine storm in a subject in need thereof comprising administering an effective amount of a cannabinoid to treat or reduce the cytokine storm. In one embodiment the cannabinoid is cannabichromene. In other embodiments, the cannabinoid is selected from the group consisting of tetrahydrocannabinols (THC), preferably delta-9-tetrahydrocannabinol and delta-8-tetrahydrocannabinol, cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV) and cannabigerol monomethyl ether (CBGM), arachidonoylethanolamine (AEA), 2-arachidonoylglycerol (2-AG), 2-arachidonyl glyceryl ether (noladin ether), N-arachidonoyl dopamine (NADA), virodhamine (OAE) lysophosphatidylinositol (LPI), nabilone, rimonabant, JWH-073, CP-55940, dimethylheptylpyran, HU-210, HU-331, SR144528, WIN 55,212-2, JWH-133, levonantradol, and AM-2201 and combinations thereof.

### BRIEF DESCRIPTION OF THE DRAWINGS

FIGS. 1A-1Y show that inhalant cannabichromene (CBC) treatment modulated TRPs expression and improved symptoms of ARDS, limiting cytokine storm in both blood and lung tissues.

FIG. 1A is an illustration of the inhaler setting using ApelinDxtm Inhaler for delivery of inhalant CBC to the mice.

FIG. 1B is a bar graph showing blood oxygen saturation levels in mice treated with Poly (I:C) followed by the treatment of inhalant CBC (PIC+CBC) compared to untreated group (PIC).

FIGS. 1C-1H are panels showing histological analysis, Hematoxylin and Eosin (H&E, 1C-1E) as well as Masson's trichrome (MT, 1F-1H) demonstrating the effect of Poly (I:C) on the morphology and architecture of lung in mice (PIC) compared to the normal control group (sham).

FIG. 1I-1N are panels showing Immunohistochemical analysis of expression levels of TRPA1 (1I-1K) as well as TRPV1 (1L-1N) in lung tissues (arrows) of CBC treated group (PIC+CBC) compared to normal sham or Poly (I:C) treated one (Sham or PIC) (Images are all 200× and Inner images are 630× magnification).

FIGS. 1O-1T are scatter plots (1O-1Q) and line graphs (1R-1T) showing flow cytometry analysis intranasal administration of Poly(I:C) on pro-inflammatory cytokines including IL-6 (FIG. 1R), IL-17 (1S) and IFNg (FIG. 1T) compared to the control group (FIG. 1O) in whole blood.

FIG. 1U is a bar graph showing the effects of intranasal administration of Poly(I:C) on the expression levels pro-inflammatory cytokines in whole blood.

FIGS. 1V-1aa are scatter plots and line graphs showing flow cytometry analysis of the effects of intranasal administration of Poly(I:C) on pro-inflammatory cytokines including IL-6 (FIG. 1Y), IL-17 (FIG. 1Z) and IFNg (FIG. 1aa) compared to the control group (FIG. 1V).

FIG. 1ab is a bar graph showing the effects of intranasal administration of Poly(I:C) on the expression levels pro-inflammatory cytokines in the lung (**p<0.01).

FIGS. 2A-2F are dot plot flow cytometry panels showing that CBD improved the symptoms of Poly(I:C)-induced ARDS (FIG. 2E-2F) and normalized the expression level of apelin in the blood. Intranasal administration of poly(I:C) demonstrated a reduction in the blood T cells (FIG. 2C) while increased the neutrophils compared with the sham control group (FIG. 2A).

FIGS. 2G-2H are histogram and bar graph panels, respectively, showing that intranasal poly(I:C) reduced apelin expression in whole blood of mice, as assessed by flow cytometry. Administration of CBD attenuated these effects. The bar graphs are representing the average of values for 10 mice per group (**P<0.03).

FIGS. 3A-3F are panels showing Masson's trichrome analysis of lung tissues where intranasal administration of high dose Poly(I:C) caused the destruction of normal morphology and structure of lung, hypertrophy, fibrosis and pulmonary oedema (FIGS. 3C-3D), as compared to the tissues from control group (FIGS. 3A-3B). CBD treatment improved the structure towards the normal architecture (FIGS. 3E-3F).

FIGS. 3G-3R are panels showing the immunofluorescence analysis of lung tissue which showed a decrease in Apelin in Poly(I:C) treated lung (FIG. 3I) compared to normal tissues (FIG. 3G). CBD treatment normalized the Apelin expression in the lung, indicating the potential protective effect of CBD (FIG. 3K).

### DETAILED DESCRIPTION OF THE INVENTION

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, “cannabichromene (CBC)” refers to the major nonpsychotropic cannabinoid compound derived from the plant Cannabis sativa, commonly known as marijuana.

The term “coronavirus” refers to a group of related RNA viruses that cause diseases in mammals and birds. In humans, these viruses cause respiratory tract infections that can range from mild to lethal. Mild illnesses include some cases of the common cold (which is caused also by certain other viruses, predominantly rhinoviruses), while more lethal varieties can cause SARS, MERS, and COVID-19. There are presently no vaccines or antiviral drugs to prevent or treat human coronavirus infections.

The term “SARS” or “severe acute respiratory syndrome” refers to a viral respiratory disease of zoonotic origin that surfaced in the early 2000s caused by severe acute respiratory syndrome coronavirus (SARS-CoV or SARS-CoV-1), the first-identified strain of the SARS coronavirus species severe acute respiratory syndrome-related coronavirus (SARSr-CoV). The syndrome caused the 2002-2004 SARS outbreak. In 2019, its successor, the related virus strain Severe Acute Respiratory Syndrome Coronavirus 2 (SARS-CoV-2), was discovered.

The term “Covid-19” or “Coronavirus disease 2019” refers to a severe acute respiratory syndrome (SARS) caused by a virus known as SARS-Coronavirus 2 (SARS-CoV2).

The terms “treating” or “treatment” as used herein with reference to therapeutic uses of compounds of describe herein for the management or care of a patient for the purposes of combatting disease, and includes the administration of the active agent to asymptomatic individuals, for example to prevent the onset of the symptoms or complications, i.e. prophylaxis.

The active agents are to be administered to human subjects in “therapeutically effective amounts”, which is taken to mean a dosage sufficient to provide a medically desirable result in the patient. The exact dosage and frequency of administration of a “therapeutically effective amount” of active agent will vary, depending on the condition which it is desired to treat, the stage and severity of disease, and such factors as the nature of the active substance, the dosage form and route of administration.

The term “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

A “pharmaceutical composition” can include the combination of an active agent, such as a therapeutic peptide, with a carrier, inert or active, in a sterile composition suitable for diagnostic or therapeutic use in vitro, in vivo or ex vivo.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically acceptable composition” as used herein refers to a composition comprising at least one compound as disclosed herein formulated together with one or more pharmaceutically acceptable carriers.

The term “administration” refers to the introduction of an amount of a predetermined substance into a patient by a certain suitable method. The composition disclosed herein may be administered via any of the common routes, as long as it is able to reach a desired tissue, for example, but is not limited to, inhaling, intraperitoneal, intravenous, intramuscular, subcutaneous, intradermal, oral, topical, intranasal, intrapulmonary, or intrarectal administration. However, since peptides are digested upon oral administration, active ingredients of a composition for oral administration should be coated or formulated for protection against degradation in the stomach.

The term “dose” means a single amount of a compound or an agent that is being administered thereto; and/or “regimen: which means a plurality of pre-determined doses that can be different in amounts or similar, given at various time intervals, which can be different or similar in terms of duration. In some embodiments, a regimen also encompasses a time of a delivery period (e.g., agent administration period, or treatment period). Alternatively, a regimen is a plurality of predetermined plurality pre-determined vaporized amounts given at pre-determined time intervals.

### II. Cannabichromene as a Therapeutic Modality for COVID-19

With a limited number of effective antivirals, the pandemic of COVID-19 remains the most significant challenge to the healthcare system in decades. There is an urgent need for definitive therapeutic intervention. Clinical reports indicate the cytokine storm associated with acute respiratory distress syndrome (ARDS) is the leading cause of mortality in severe cases of COVID-19. Disclosed herein are cannabichromene (CBC) compositions and methods of their use as a therapeutic modality for COVID-19.

A. Cannabinoid Compositions

The term “cannabinoid” as used herein may encompass a chemical compound that activates any mammalian cannabinoid receptor, for example human CB₁ receptor or human CB2receptor. The cannabinoids may be naturally occurring (such as, for example, endocannabinoids or phytocannabinoids) or they may be synthetic. Synthetic cannabinoids may include, for example, the classical cannabinoids structurally related to THC, the non-classical cannabinoids (cannabimimetics) including the aminoalkyindoles, 1,5-diarylpyrazoles, quinolines and arylsulphonoamides, and eicosanoids related to the endocannabinoids. When a cannabinoid salt is used, it may be employed in the form of a solution. The one or more cannabinoids is preferably selected from the classical cannabinoids, more preferably selected from tetrahydrocannabinols (THC), preferably delta-9-tetrahydrocannabinol and delta-8-tetrahydrocannabinol, cannabidiol (CBD), cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV and cannabigerol monomethyl ether (CBGM). CBC is a preferred cannabinoid.

Cannabichromene (CBC) is a non-psychotropic phytocannabinoid that offers several of the benefits that THC does while not having such pronounced effects. Studies have shown that CBC does not interact significantly with CB1 receptors in the body, but that it does have some interactions with the CB2 receptors. This fact may be why it does not produce much of a psychoactive effect. Early reports showed that CBC prolonged hexobarbital hypnosis in mice (Hatoum et al., 1981) exerted anti-inflammatory effects and modest analgesic activity in rodents (Wirth et al., 1980; Turner and Elsohly, 1981; Davis and Hatoum, 1983), while showing no ‘Cannabis like’ activity in the Rheseus monkey (Mechoulam et al., 1970) and in human smoking experiments (Turner et al., 1980). In more recent years, it has been shown that CBC exerts antimicrobial (Appendino et al., 2008), anti-inflammatory (DeLong et al., 2010; Tubaro et al., 2010), analgesic (Maione et al., 2011) and antidepressant-like activity in rodents (El-Alfy et al., 2010). Pharmacodynamic studies have shown that CBC, like other plant natural products (Gertsch et al., 2010), is an inhibitor of endocannabinoid cellular reuptake (Ligresti et al., 2006) and a weak inhibitor of monoacylglycerol lipase (MAGL) (De Petrocellis et al., 2011), but is also a potent activator of transient receptor potential (TRP) ankyrin 1-type (TRPA1) channels (De Petrocellis et al., 2008; 2011).

Other cannabinoids suitable for use in the present invention are endocannabinoids, substances that naturally occur in the mammalian body and which activate one or more cannabinoid receptor. Preferably endocannabinoids are selected from arachidonoylethanolamine (AEA), 2-arachidonoylglycerol (2-AG), 2-arachidonyl glyceryl ether (noladin ether), N-arachidonoyl dopamine (NADA), virodhamine (OAE) and lysophosphatidylinositol (LPI).

Synthetic cannabinoids suitable for use in the present invention include nabilone, rimonabant, JWH-073, CP-55940, dimethylheptylpyran, HU-210, HU-331, SR144528, WIN 55,212-2, JWH-133, levonantradol, and AM-2201.

In recent years, cannabinoids have been investigated extensively due to their potential effects on the human body. Among all cannabinoids, Cannabichromene (CBC)has demonstrated a potent anti-inflammatory effect in a variety of inflammatory conditions. In one embodiment, CBC is used to contain the cytokine storm and treat the cytokine release syndrome associated with COVID-19 and other inflammatory viral conditions.

In another embodiment, the administration of CBC downregulates the level of pro-inflammatory cytokines and ameliorates the clinical symptoms of COVID-19. One embodiment provides a therapeutic role for CBC in the treatment of COVID-19 by reducing the cytokine storm, containing the damage, and re-establishing homeostasis.

B. Intranasal Compositions

In one embodiment, the cannabichromene compositions are formulated to allow intranasal administration. Intranasal compositions may comprise an inhalable dry powder pharmaceutical formulation comprising a therapeutic agent, wherein the therapeutic agent is present as a freebase or as a mixture of a salt and a freebase. Pharmaceutical formulations disclosed herein can be formulated as suitable for airway administration, for example, nasal, intranasal, sinusoidal, peroral, and/or pulmonary administration. Typically, formulations are produced such that they have an appropriate particle size for the route, or target, of airway administration. As such, the formulations disclosed herein can be produced so as to be of defined particle size distribution.

For example, the particle size distribution for a salt form of a therapeutic agent for intranasal administration can be between about 5 μm and about 350 μm. More particularly, the salt form of the therapeutic agent can have a particle size distribution for intranasal administration between about 5μ to about 250 μm, about 10 μm to about 200 μm, about 15 μm to about 150 μm, about 20 μm to about 100 μm, about 38 μm to about 100 μm, about 53 μm to about 100, about 53 μm to about 150 μm, or about 20 μm to about 53 μm. The salt form of the therapeutic agent in the pharmaceutical compositions of the invention can a particle size distribution range for intranasal administration that is less than about 200 μm. In other embodiments, the salt form of the therapeutic agent in the pharmaceutical compositions has a particle size distribution that is less than about 150 μm, less than about 100 μm, less than about 53 μm, less than about 38 μm, less than about 20 μm, less than about 10 μm, or less than about 5 μm. The salt form of the therapeutic agent in the pharmaceutical compositions of the invention can have a particle size distribution range for intranasal administration that is greater than about 5 μm, greater than about 10 μm, greater than about 15 μm, greater than about 20 μm, greater than about 38 μm, less than about 53 μm, less than about 70 μm, greater than about 100 μm, or greater than about 150 μm.

Additionally, the salt form of the therapeutic agent in the pharmaceutical compositions of the invention can have a particle size distribution range for pulmonary administration between about 1 μm and about 10 μm. In other embodiments for pulmonary administration, particle size distribution range is between about 1 μm and about 5 μm, or about 2 μm and about 5 μm. In other embodiments, the salt form of the therapeutic agent has a mean particle size of at least 1 μm, at least 2 μm, at least 3 μm, at least 4 μm, at least 5 μm, at least 10 μm, at least 20 μm, at least 25 μm, at least 30 μm, at least 40 μm, at least 50 μm, at least 60 μm, at least 70 μm, at least 80 μm, at least 90 μm, or at least 100 μm.

In some embodiments the disclosed cannabinoid compositions include one or more cannabinoids or pharmaceutically acceptable derivatives or salts thereof, a propellant, an alcohol, and a glycol and/or glycol ether. The alcohol may be a monohydric alcohol or a polyhydric alcohol, and is preferably a monohydric alcohol. Monohydric alcohol has a lower viscosity than a glycol or glycol ether. Accordingly, the composition is able to form droplets of a smaller diameter in comparison to compositions in which the monohydric alcohol is not present. The present inventors have surprisingly found that a specific ratio of monohydric alcohol to glycol or glycol ether results in a composition with a desired combination of both long term stability (for example the composition remains as a single phase for at least a week at a temperature of 2-40° C.) and small droplet size.

C. Pulmonary Compositions

One embodiment provides a formulation and method for treating ARDS in the pulmonary system by inhalation or pulmonary administration. The diffusion characteristics of the particular drug formulation through the pulmonary tissues are chosen to obtain an efficacious concentration and an efficacious residence time in the tissue to be treated. Doses may be escalated or reduced or given more or less frequently to achieve selected blood levels. Additionally, the timing of administration and amount of the formulation is preferably controlled to optimize the therapeutic effects of the administered formulation on the tissue to be treated and/or titrate to a specific blood level.

Diffusion through the pulmonary tissues can additionally be modified by various excipients that can be added to the formulation to slow or accelerate the absorption of drugs into the pulmonary tissues. For example, the drug may be combined with surfactants such as the phospholipids, dimyristoylphosphatidyl choline, and dimyristoylphosphatidyl glycerol. The drugs may also be used in conjunction with bronchodilators that can relax the bronchial airways and allow easier entry of the antineoplastic drug to the lung. Albuterol is an example of the latter with many others known in the art. Further, the drug may be complexed with biocompatible polymers, micelle forming structures or cyclodextrins.

Particle size for the aerosolized drug used in the present examples was measured at about 1.0-5.0 μm with a GSD less than about 2.0 for deposition within the central and peripheral compartments of the lung. As noted elsewhere herein particle sizes are selected depending on the site of desired deposition of the drug particles within the respiratory tract.

Aerosols useful in the invention include aqueous vehicles such as water or saline with or without ethanol and may contain preservatives or antimicrobial agents such as benzalkonium chloride, paraben, and the like, and/or stabilizing agents such as polyethyleneglycol.

Powders useful in the invention include formulations of the neat drug or formulations of the drug combined with excipients or carriers such as mannitol, lactose, or other sugars. The powders used herein are effectively suspended in a carrier gas for administration. Alternatively, the powder may be dispersed in a chamber containing a gas or gas mixture which is then inhaled by the patient.

### III. Apelin

Apelin, an endogenous, multi-functional ligand for the G protein-coupled receptor, APJ, also serves as a second catalytic substrate for ACE2 (Chen. L J, et al., Int J Hypertens. 2015:5 (2015)). Apelin is generated from a 77-amino acid precursor and undergoes proteolytic cleavage to generate biological active fragments, including apelin-36, apelin-19 and apelin-13. An endogenous protective role was postulated for activation of the apelin/APJ axis (Apelinergic system) after lung injury, via proposed mechanisms including suppression of the immune activating transcription factor, NF-κB and inhibition of innate immune infiltration/activation via attenuated expression of CCL2, CCL3, CCL4, CCL7 and TNF-α (Huang, S., et al., Clin Chim Acta., 456:81 (2016)). Of interest, both apelin and APJ are widely expressed throughout the lung, heart, liver, gut, kidney and central nervous system (Kawamata, Y., et al., Biochim Biophys Acta., 1538(2-3):162 (2001)), spatially overlapping expression of the endocannabinoid system while interaction between the endocannabinoid system and apelin limits liver fibrosis (Melgar-Lesmes, P., et al., Cells, 8:1311 (2019)). In one embodiment the regulation of the apelinergic system by CBC limits excessive pulmonary inflammation after ARDS.

### IV. Administration

Inhalation is a convenient administration route for therapeutic agents that overcomes many of the drawbacks of oral administration, such as slow drug onset and first-pass metabolism plus it can be used with patients that suffer from pulmonary conditions.

A. Intranasal Administration

In one embodiment, the CBC compositions are delivered through intranasal administration. As described herein, intranasal administration or nose administration comprise the described compositions being administered into the mammal nostril and reaching nasal meatus or nasal cavity. For example, the compositions can be administered with nasal spray, insufflation, nasal drop, aerosol, propellant, pressurized dispersion body, aqueous aerosol, propellant, nose suspension, instillation, nasal gel, nose is with ointment and nose ointment, by means of any new or old type equipment of administration.

Current studies strongly suggest that intranasal administration of high dose Polyinosinic:polycytidylic acid (Poly(I:C)) in a murine model maybe a reliable working and practical model to investigate and help better understanding of mechanisms responsible for COVID-19 symptoms. Induction of hypo-oxygenation, lymphopenia, cytokine storm (marked production of proinflammatory cytokines) as well as impairment and destruction of lung architecture are all examples of cardinal clinical symptoms of severe COVID-19 and ARDS. In addition, being a synthetic analog of double stranded RNA, not only can Poly(I:C) provide a very robust foundation to study the immunopathology and physiology of COVID-19, but when compared to many viral agents, it is considerably safer and cheaper alternative for research on SARS-Cov-2 and other virus-induced infections.

Present findings propose a potential immunotherapeutic role for CBC in the treatment of severe respiratory viral infections and ARDS. The current data support the notion that anti-inflammatory function of CBC may be a crucial modulator of cytokine storm and hyperinflammation. There are reports suggesting that the interaction between immune system and COVID-19 is a two-phased process. The first stage is the activation of immune system to decimate the viruses and contain the progression of infection. The second stage is characterized by a regulatory mechanism to curtail the cytokine storm and prevent the cytokine-induced sepsis. The vast distribution of endocannabinoid system in the body is well documented and mounting evidence support anti-inflammatory effects for cannabinoids. Therefore, altogether, it maybe plausible to propose CBC as a potential immunomodulator in the treatment of COVID-19 and ARDS. In conclusion, these new findings introduce a new angle with a translational perspective to investigate the potentials of cannabinoids in the treatment of viral respiratory diseases such as COVID-19. Further studies are required to foster and validate such a complex therapeutic strategy in the treatment of severe viral respiratory infections such as COVID-19.

One embodiment provides a method of treating COVID-19 symptoms in a subject in need thereof by administering to the subject an effective amount of a composition including cannabichromene.

Another embodiment provides a method of reducing Acute respiratory distress syndrome in a subject in need thereof by administering to the subject an amount of cannabichromene effective to reduce inflammation in the subject. The cannabichromene reduces the level of inflammatory cytokines. The cytokines can be circulating cytokines or lung cytokines. In one embodiment, cannabichromene treatment reduces inflammatory damage to the lungs. In another embodiment, cannabichromene treatment improves the functional capacity of the lungs. Acute respiratory distress syndrome can be caused by COVID-19.

B. Pulmonary Administration

In another embodiment, the cannabinoids are delivered through pulmonary administration directly to the lungs where they are efficiently absorbed into the systemic circulation, resulting in a rapid onset of therapeutic action. The rapid onset of therapeutic action achievable through the compositions and methods of the invention offers an advantage over prior cannabinoid delivery methods such as sublingual or suppository delivery, which generally involve slower systemic absorption.

Pulmonary administration by inhalation may be accomplished by means of producing liquid or powdered aerosols, for example, by using any of various devices known in the art. PCT Publication No. WO 92/16192 dated Oct. 1, 1992; PCT Publication No. WO 91/08760 dated Jun. 27, 1991; NTIS Patent Application 7-504-047 filed Apr. 3, 1990 by Roosdorp and Crystal) including but not limited to nebulizers, metered dose inhalers, and powder inhalers. Various delivery devices are commercially available and can be employed, e.g. Ultravent nebulizer (Mallinckrodt, Inc, St. Louis, Mo.); Acorn II nebulizer (Marquest Medical Products, Englewood, Colo.); Ventolin metered dose inhalers (Glaxo Inc., Research Triangle Park, N.C.); Spinhaler powder inhaler (Fisons Corp., Bedford, Mass.) or Turbohaler (Astra). Such devices typically entail the use of formulations suitable for dispensing from such a device, in which a propellant material may be present. Ultrasonic nebulizers may also be used.

As will be understood by those skilled in the art of delivering pharmaceuticals by the pulmonary route, a major criterion for the selection of a particular device for producing an aerosol is the size of the resultant aerosol particles. Smaller particles are needed if the drug particles are mainly or only intended to be delivered to the peripheral lung, i.e. the alveoli (e.g. 0.1-3 μm), while larger drug particles are needed (e.g. 3-10 μm) if delivery is only or mainly to the central pulmonary system such as the upper bronchi. Impact of particle sizes on the site of deposition within the respiratory tract is generally known to those skilled in the art.

### EXAMPLES

### Example 1. Cannabichromene as a Treatment Modality for COVID-19

Materials and Methods:

Animal model and application of Poly(I:C) and CBC: Wild-type (WT) C57BL/6 mice (male, 12 weeks old) were divided into 3 experimental groups of sham, control and treatment (n=5). All animals were housed in pathogen-free conditions at the animal facility of the Augusta University, and all experiments were performed in accordance with the rules and regulations of the Augusta University Institutional Animal Care and Use Committee (IACUC). All mice were anesthetized with isoflurane. Sham group received PBS while control and treatment groups were administered by Poly(I:C) (Sigma Aldrich, USA) (100 μg in 50 μl sterile PBS) intranasally (I/N) three once-daily doses. CBC (isolate THC free) was delivered intraperitoneally (5 mg/kg), first dose two hours after the second Poly(I:C) treatment and every other day interval, total of 3 doses. Sham and control groups received PBS only. All mice were sacrificed at 8 days after the first Poly(I:C) application. Blood and lung tissues were harvested and subjected to the further analysis.

Measurement of vital signs: Vital signs including temperature, Blood O₂ saturation were measured prior and post of any treatment. Central body temperature was measured rectally and blood oxygen saturation was determined using portable pulse oximetry through carotid arteries.

Histology and immunohistochemistry: Left lobes of lung tissue were fixed in 10% neutral buffered formalin. Samples were processed by routine methods, oriented so as to provide coronal sections and 5 micron mid-coronal sections cut and stained with hematoxylin & eosin, Trichrome for histology. As for inflammatory indices, immunohistochemistry was performed by incubating the samples with specific antibodies against murine IL-6 (Cat #554402, BD BioSciences Pharmingen) and Neutrophils (Cat #G102, Leinco Technologies). Preparations were counterstained with hematoxylin (catalog no. 7221; Richard-Allan Scientific, Kalamazoo, Mich., USA) and mounted in Faramount (catalog no. S3025, DAKO), analyzed and imaged by brightfield microscopy.

Analytical Flow Cytometry: Single cell suspension was prepared from lung tissues. Briefly, tissue samples were sieved through a 100 μM cell strainer (BD Biosciences, San Diego, Calif.), followed by centrifugation (1,000 rpm, 10 min) to prepare single-cell suspensions. All cells were then stained with fluorescent antibodies to quantify neutrophils, macrophages, lymphocytes and cytokine expression. Briefly, all cells were stained with anti-Gr1 (Neutrophils), Anti-F4/80 (Macrophages), and anti-CD3/CD4/CD8 (Lymphocytes, all from Biolegend USA). Then cells were fixed and permeabilized and stained intracellularly for cytokines including IL-6, TNFα, IL-2, and IFNγ (Proinflammatory cytokines). All samples were run through a 4-Laser LSR II flow cytometer. Cells were gated based on forward and side scatter properties and on marker combinations to select cells of interest. All acquired flow cytometry data were analyzed using the FlowJo V10.

Statistical analysis: Graphs and summary statistics were also used to assess the results. All statistical tests were 2-sided. Except for where noted, all p-values presented are unadjusted for multiple comparisons.

Results:

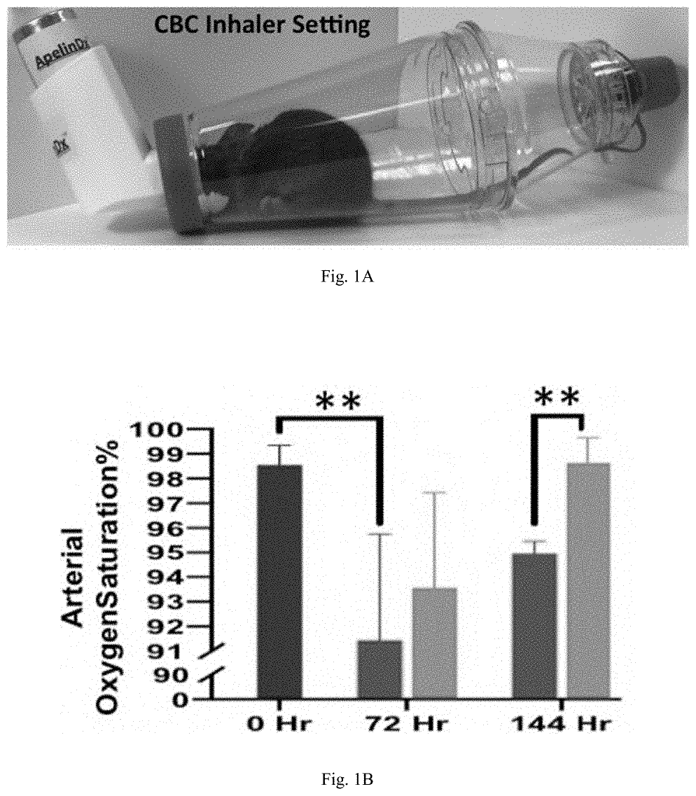

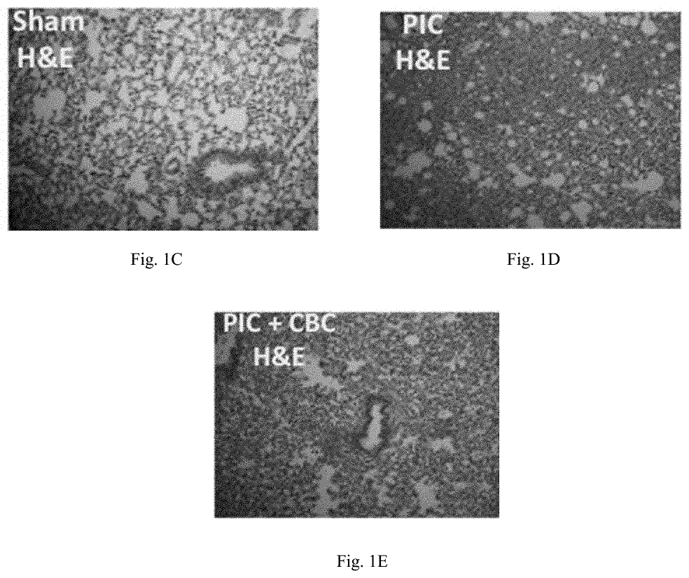

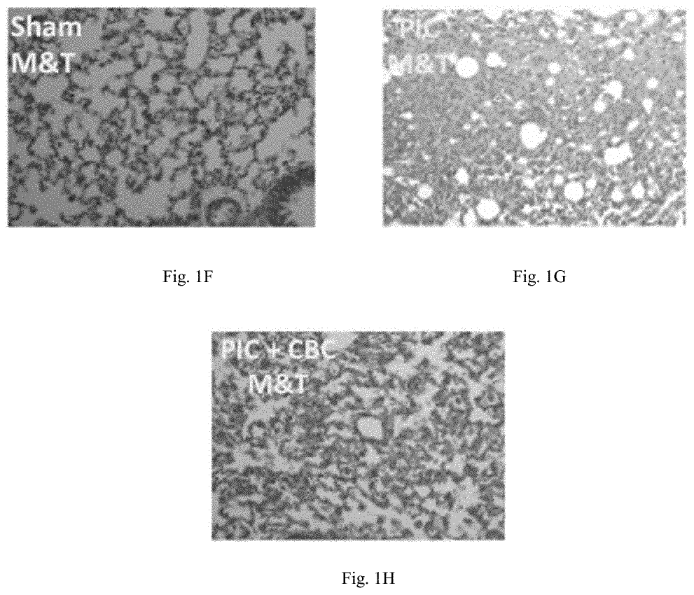

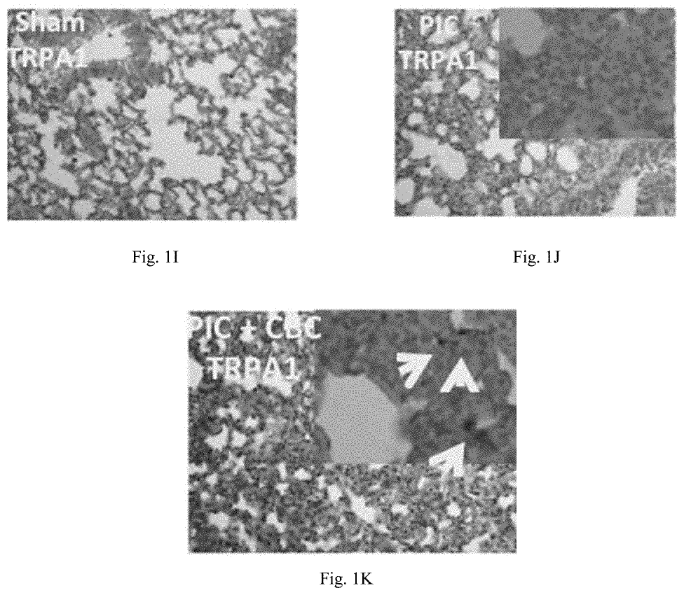

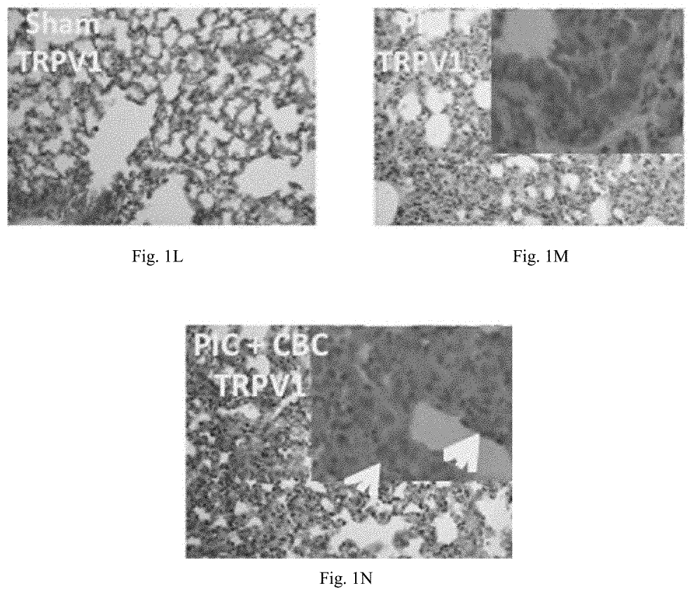

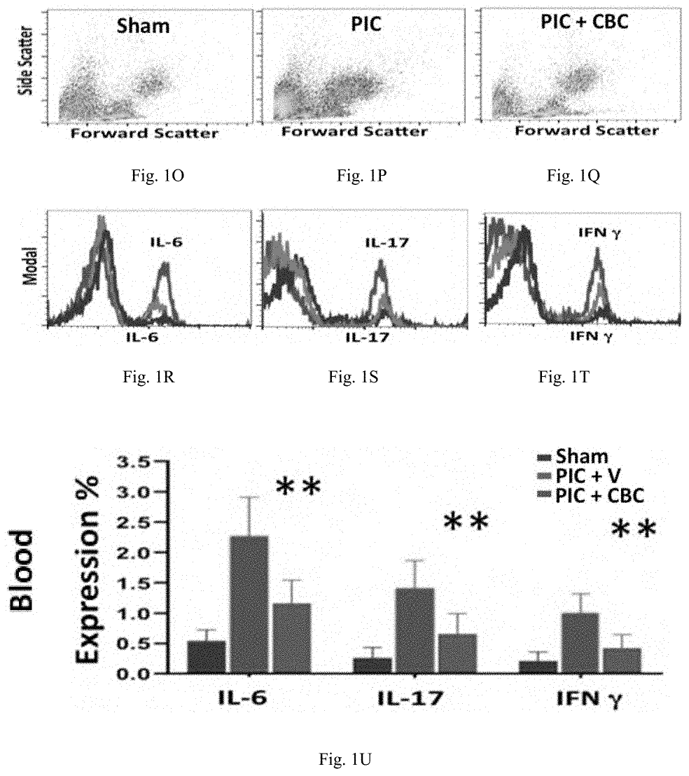

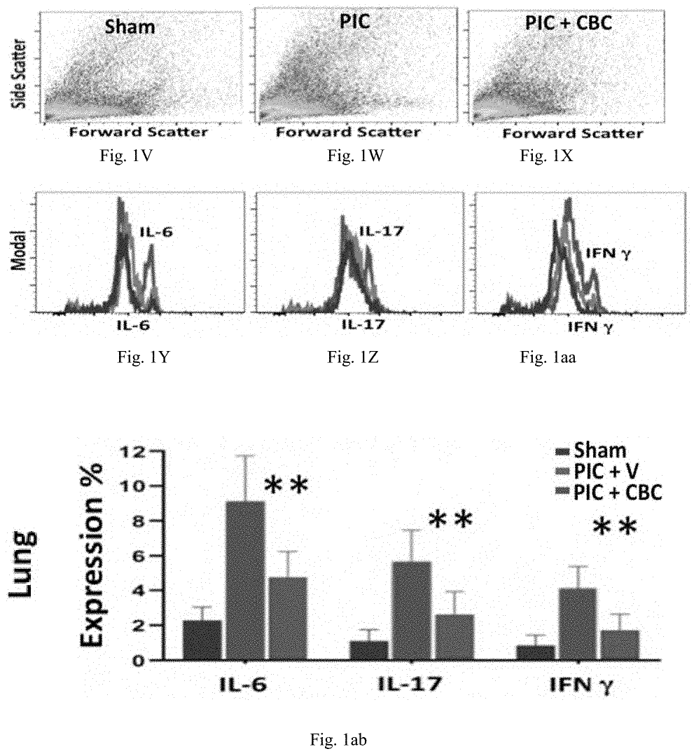

A ApelinDxtm Inhaler was connected to a spacer, providing a completely sealed, soft, and comfortable delivery of inhalant CBC to the mice (FIG. 1A). CBC treatment reversed hypoxia, increasing blood oxygen saturation positively towards the normal level by 8% from 90% +/− to 98% +/− (FIG. 1B). Histological examination of lung tissues demonstrated that Poly (I:C) caused the destruction of morphology and architecture of lung in mice (PIC) compared to the normal control group (sham). Inhalant CBC treatment reduced the ARDS-like symptoms and ameliorated the structural damages to the lung, evidenced by reduced hypertrophy and fibrosis in the lung tissues with ARDS symptoms (FIGS. 1C-1H). Further histological examination demonstrated that CBC treatment increased the level of expression for transient receptor potential (TRP) channels of vanilloid type-1 (TRPV1) and of ankyrin type-1 (TRPA1), suggesting an active interaction between CBC and TRPs (FIG. 1I-1N). Flow cytometry analysis of blood and lung demonstrated that CBC treatment curtailed the expression levels of proinflammatory cytokines including IL-6, IL-17 and IFNγ in both tissues compared to the untreated counterparts (p<0.01) (FIG. 10-1ab).

The data presented herein, demonstrates that inhalant CBC protected lung structure, contained excessive cytokine production, and curtailed inflammatory responses both in lung and blood tissues in an experimental ARDS model. The beneficial effects of inhalant CBC were correlated with activation of TRPA1 and TRPV1 cation channels, supporting the notion of potential interactions between CBC and TRPA1 as well as TRPV1. Importantly, delivery of CBC through an inhaler increases the translational value of this study and potential for trials in a clinical setting. This disclosure shows the beneficial effects of cross-talk between CBC and TRPs in ARDS.

### Example 2. CBD Improved the Symptoms of Poly(I:C)-Induced ARDS and Normalized the Expression Level of Apelin in the Blood

Materials and Methods:

Animal model and application of Poly(I:C) and CBC: Adult (12 weeks) male C57B1/6 mice were block randomized into one of three experimental groups (n=10 mice/group) by a blinded investigator. Group I received intranasal, once daily administration of sterile saline for three consecutive days to serve as a control. Group II received intranasal, once daily administration of Poly I:C (100 μg in 50 μL in sterile saline) for three consecutive days to mimic ARDS. Group III received intranasal, once daily administration of Poly I:C (100 μg in 50 μL in sterile saline) for three consecutive days, with intraperitoneal administration of CBD (isolate CBD, THC-free, 5 mg/kg body weight, Canabidiol Ltd, Dublin, Ireland), first dose two hours after the second Poly(I:C) treatment and every other day for a total of 3 doses to the treatment group. Blood oxygen saturation was quantified via the carotid arteries using a portable pulse oximeter at study initiation (day 0) and once daily for the duration of the study. Mice were euthanized at study day nine. Blood and lung tissue were harvested and subjected to flow cytometry, immunofluorescence and histological analysis, as detailed above. All flow cytometry data were analysed using the FlowJo V10 software while immunofluorescence, and histological preparations were analysed and imaged by fluorescence and bright field microscopy. As for additional histological evaluation, Masson's Trichrome staining was used for the detection of collagen fibres in lung on formalin-fixed, paraffin-embedded sections. The collagen fibres stained in blue and the background is stained red. Sections were examined and analysed using bright field microscopy imaging.

Results:

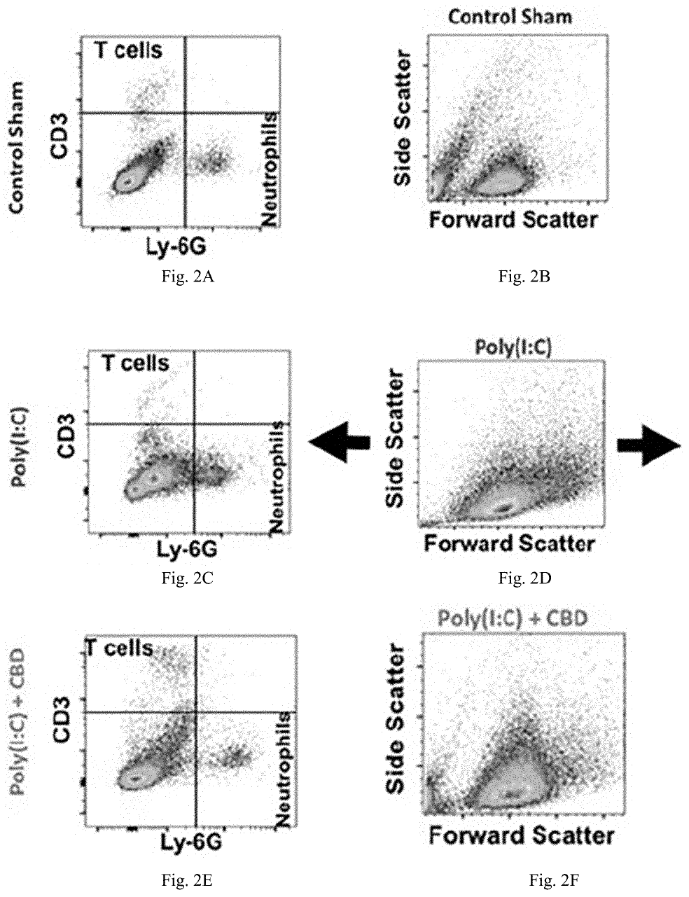

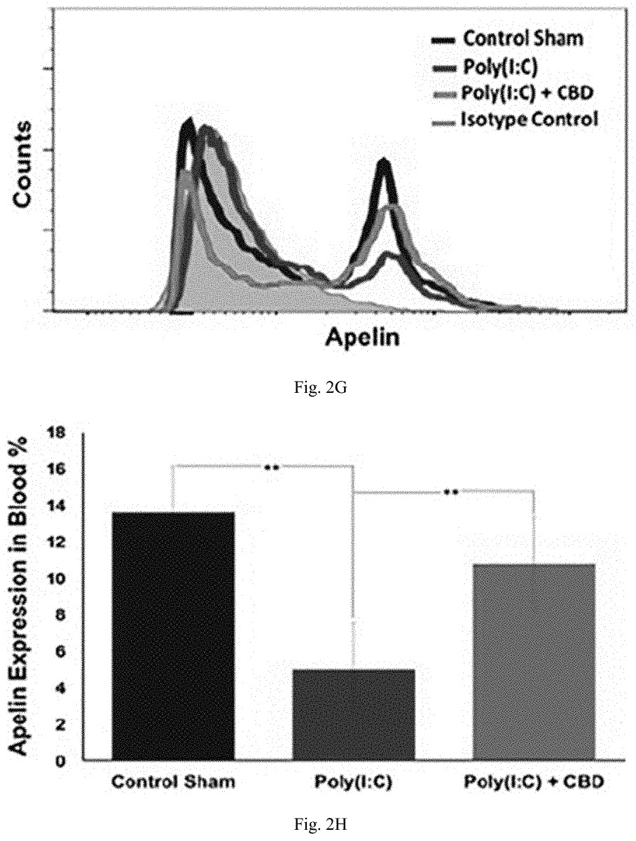

Flow cytometry analysis of whole blood showed that Poly(I:C)-treated mice exhibited a pattern of lymphopenia, lower frequency of T cells and elevated rate of neutrophils compared with the sham control group (FIGS. 2A-2D). Further, Poly(I:C)-treated mice demonstrated significant reduction in the expression level of Apelin compared with the sham control group (FIGS. 2G-2H). Conversely, administration of CBD not only returned decreased T cells and increased neutrophils towards the normal level, but also, enhanced expression of apelin in the blood following poly I:C treatment (FIGS. 2E-2F; 2G-2H). The disclosure presents a new therapeutic role for CBC in the treatment of ARDS as well as a wide range of respiratory diseases including a potential for COVID-19 and other inflammatory conditions. The use of inhalant CBC and direct delivery of CBC to the lungs offers a more rapid onset of action, allows smaller doses to be used and has a better efficacy to safety ratio compared to systemic therapy. Further, as TRPs are emerging as novel therapeutic targets through their potential roles in cellular interactions and tissue homeostasis, therefore, it is plausible to envision more prominent roles for CBC in tissue homeostasis, modulation of inflammatory responses and orchestrating a set of cellular interactions as an immunotherapeutic target, warranting further research.

### Example 3. CBD Improved the Symptoms of Poly(I:C)-Induced ARDS and Normalized the Apelin Expression in the Lung Tissues

Materials and Methods:

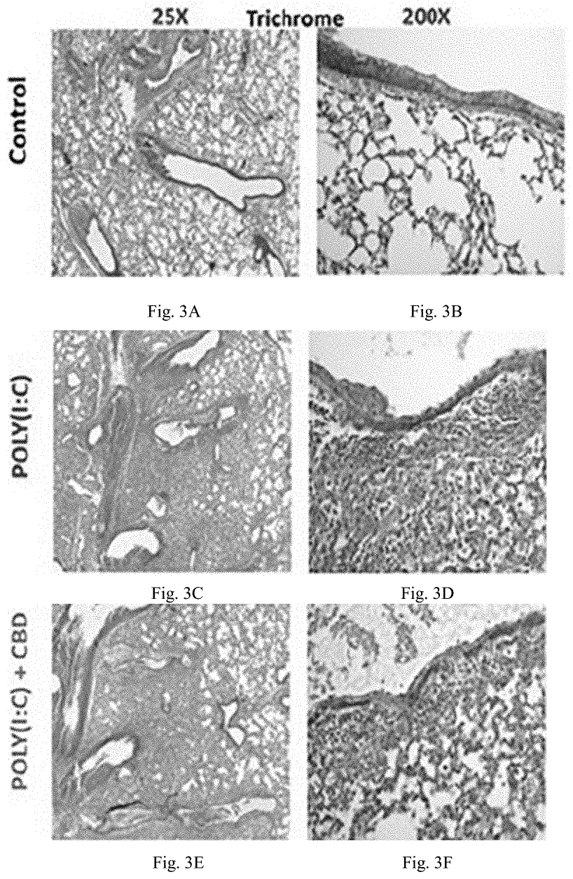

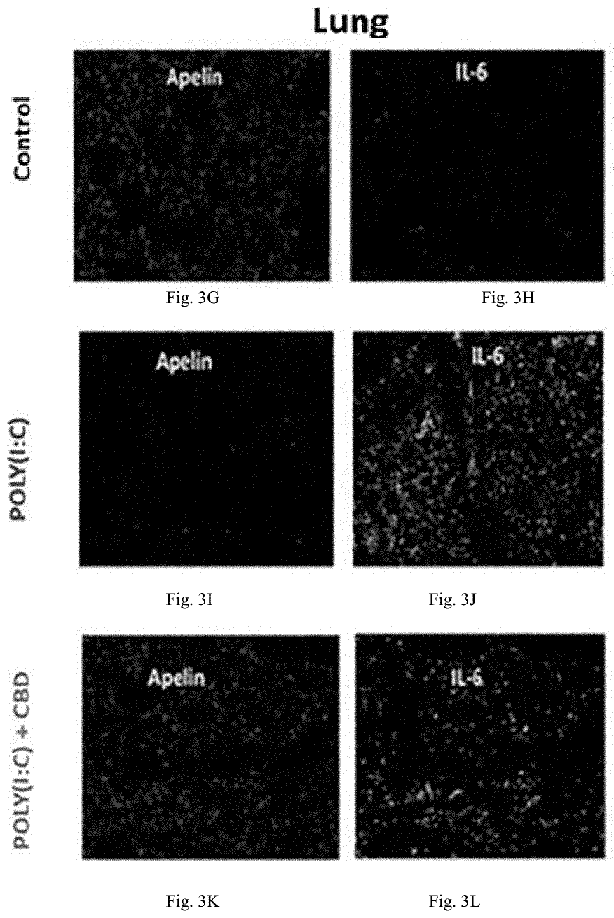

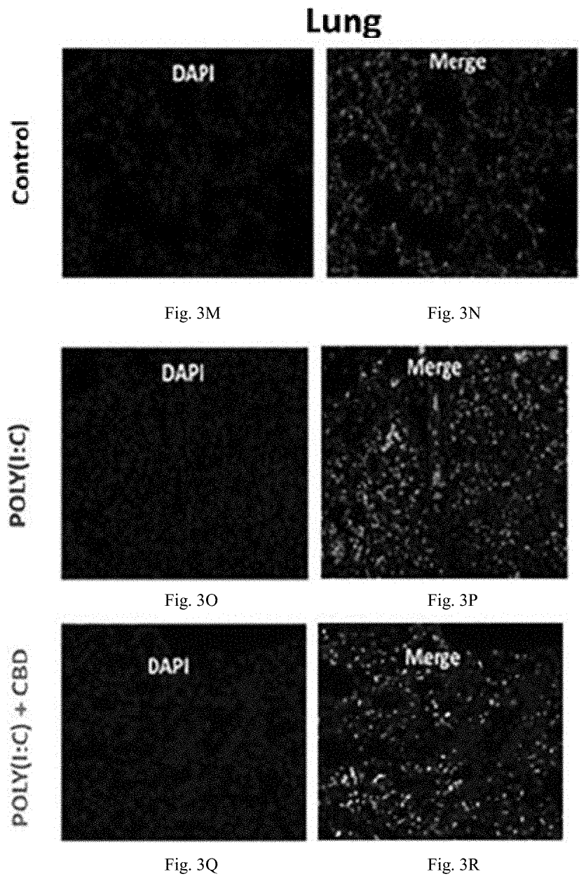

Histological examination of lung tissues demonstrated that Poly(I:C) caused significant perivascular and peri-bronchiolar interstitial inflammatory infiltrate, fibrosis, hypertrophy and pulmonary oedema, as evidenced by the widened interstitial space surrounding the airways and vasculature (FIGS. 3C-3D). The pathological features of poly I:C administration were completely or partially abolished by following administration of CBD (FIGS. 3E-3F). Immunofluorescence analysis of lung tissue revealed a reduction in apelin immunoreactivity after poly I:C treatment (FIG. 3I), as compared to control mice (FIG. 3G). Importantly, treatment with CBD increased apelin expression towards control levels in the lung following poly I:C administration (FIG. 3K).

CBD improved lung structure and exerted a potent anti-inflammatory effect following experimental ARDS. The beneficial effects of CBD were correlated with the regulation of apelin, an endogenous peptide with protective effects in pulmonary tissue.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

1. A method of reducing inflammatory symptoms of COVID-19 in a subject in need thereof comprising administering to the subject an amount of cannabichromene effective to reduce inflammation in the subject.

2. (canceled)

3. The method of claim 1, wherein the cannabichromene reduces the level of inflammatory cytokines in the subject compared to cytokine levels prior to administration of cannabichromene.

4. The method of claim 3, wherein the inflammatory cytokines are selected from the group consisting of IL-6, IFNγ and TNFα.

5. The method of claim 3, wherein the inflammatory cytokines are in the lung or airways.

6. The method of claim 1, wherein the cannabichromene treatment reduces inflammatory damage to the lungs.

7. The method of claim 1, wherein the cannabichromene treatment improves the functional capacity of the lungs.

8. (canceled)

9. A pharmaceutical composition comprising an effective amount of a cannabinoid to reduce inflammation in a subject in need thereof.

10. The pharmaceutical composition of claim 9, wherein the composition is formulated for pulmonary administration.

11. The pharmaceutical composition of claim 9, wherein the composition is formulated for nasal administration.

12. The pharmaceutical composition of claim 9, wherein the composition is formulated for aerosol administration.

13. The pharmaceutical composition of claim 9, wherein the cannabinoid is cannabichromene.

14. The pharmaceutical composition of claim 9, wherein the cannabinoid is selected from the group consisting of tetrahydrocannabinols (THC), delta-9-tetrahydrocannabinol and delta-8-tetrahydrocannabinol, cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV), cannabigerol monomethyl ether (CBGM), arachidonoylethanolamine (AEA), 2-arachidonoylglycerol (2-AG), 2-arachidonyl glyceryl ether (noladin ether), N-arachidonoyl dopamine (NADA), virodhamine (OAE) lysophosphatidylinositol (LPI), nabilone, rimonabant, JWH-073, CP-55940, dimethylheptylpyran, HU-210, HU-331, SR144528, WIN 55,212-2, JWH-133, levonantradol, and AM-2201 and combinations thereof.

15. A method of reducing pulmonary inflammation in a subject in need thereof comprising administering to the subject an effective amount of a cannabinoid to the pulmonary inflammation.

16. The method of claim 15, wherein the cannabinoid is cannabichromene.

17. The method of claim 15, wherein the cannabinoid is selected from the group consisting of tetrahydrocannabinols (THC), preferably delta-9-tetrahydrocannabinol and delta-8-tetrahydrocannabinol, cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV) and cannabigerol monomethyl ether (CBGM), arachidonoylethanolamine (AEA), 2-arachidonoylglycerol (2-AG), 2-arachidonyl glyceryl ether (noladin ether), N-arachidonoyl dopamine (NADA), virodhamine (OAE) lysophosphatidylinositol (LPI), nabilone, rimonabant, JWH-073, CP-55940, dimethylheptylpyran, HU-210, HU-331, SR144528, WIN 55,212-2, JWH-133, levonantradol, and AM-2201 and combinations thereof.

18. The method of claim 15, wherein the subject in need thereof is infected with a coronavirus.

19. The method of claim 18, wherein the coronavirus is SARS-CoV-2.

20. The method of claim 15, wherein the subject in need thereof has ARDS.

21.-25. (canceled)
